(19) United States
(12) Patent Application Publication
(10) Pub. No.: US 2022/0339112 A1
(43) Pub. Date: Oct. 27, 2022

(54) TARGETED CHEMICAL DELIVERY CAPSULE

(71) Applicant: Patrick Harris, Shorewood, IL (US)

(72) Inventor: Patrick Harris, Shorewood, IL (US)

(21) Appl. No.: 17/711,948

(22) Filed: Apr. 1, 2022

Related U.S. Application Data
(60) Provisional application No. 63/172,394, filed on Apr. 8, 2021.

(51) Int. Cl. A61K 9/48

(52) U.S. Cl. CPC A61K 9/4808

(57) ABSTRACT

An ingestible drug delivery system that allows targeted drug delivery to large snakes and reptiles while passing through mammals without delivering the drug via the difference in digestion resistance times between snakes and mammals. A crush resistant shell prevents the drug from being released prematurely by the chewing action of the mammal.

### CROSS-REFERENCE TO RELATED APPLICATION

This application claims the benefit of U.S. Provisional Patent Application No. 63/172,394 filed Apr. 8, 2021, which is hereby incorporated by reference in its entirety herein.

### BACKGROUND OF THE INVENTION

### I. Field of the Invention

The present disclosure relates generally to the reduction of specific animals in the wild, particularly to a targeted drug delivery to such animals, and more particularly to ingestible chemical or drug delivery systems therefor.

### II. Description of the Prior Art

Invasive species continue to be a growing problem, both in the US and throughout the world. An invasive species is a nonnative organism, such as a disease, parasite, plant, or animal that begins to spread or expand its range from the size of its original introduction. This expansion typically causes harm to the environment, the economy and/or human health. Costly effects include but are in no way limited to crop decimation and other adverse effects for farmers and ranchers, clogging of water facilities and waterways, threats to fisheries, increased fire vulnerability, ecosystem issues and disease transmission.

A few well-known examples of such invasive species include unintentional introduction of predator game fish, the West Nile virus, chestnut blight, the South American fire ant, zebra mussels, Burmese pythons and sea lamprey. Indeed, the southern United States has a significant problem with invasive predatory reptiles including pythons and large lizards. The current problem with Burmese Pythons in Florida is so serious that the pythons are extending their range. This is likely due to their consumption of most of the available prey in their former home range, forcing them to migrate to new hunting grounds.

Elimination of invasive Burmese Pythons in Florida has included bounties, hiring so-called “python hunters”, radio tracking male snakes to breeding females who will tend to congregate together during breeding season, and organizing intensive media promoting “Python Roundups”. These elimination methods do capture pythons, but in extremely limited numbers. Indeed, it has been determined that such methods only seem to eliminate hundreds of snakes annually. The main problem with these methods is that they are basically a one-on-one solution that requires one or more hunters or trackers per python eliminated. This results in a large amount of manpower and financial resources that need to be expedited per python eliminated.

Using acetaminophen (Tylenol®) has been long known as an effective way to poison reptiles such as snakes. For example, laced frozen mice were used on the island of Guam to reduce the population of brown tree snakes that were preying on some native bird species towards the point of extinction. Accordingly, and while invasive reptiles can be poisoned, the current practices remain problematic. Foremost, poisons that are effective on predatory reptiles are also effective on desirable native mammal predator species including Florida panthers, foxes, and even dogs and cats. For this reason, the State of Florida currently has regulations against using poisons on wildlife.

The present disclosure provides a solution to this problem through taking advantage of the difference in digestive cycle times between predatory reptiles and predatory mammals. Predatory reptiles have a relatively long digestive cycle time (e.g. several days) as compared to predatory mammals shorter time (e.g. less than one day). Accordingly, a drug delivery system that takes advantage of this difference in digestive cycle time by taking a long time to break down in the intestine would therefor pass through a panther or other such predatory carnivore mammal before the poison could be released but instead would be released in the intestine of a python or other predatory reptile due to the longer digestive cycle time of the reptile.

Enteric coating for pills and capsules ensure that the pill passes through the acidic (low pH) stomach and is only dissolved in the more basic or neutral (higher pH) of the intestines. Thus, the desired increase in initial chemical or drug release time can be accomplished by increasing the resistance of the enteric coating of the pill to the more neutral pH of the intestines and therefor taking a longer time to dissolve in the intestines.

While a large number of poisons are known to be effective against reptiles, including large snakes and lizards, the problem remains that some predatory reptiles favor consuming prey that they have killed themselves over dead prey or a piece of meat, fish or other bait material. Additionally, while the increased resistance of a enteric coating will provide the necessary delay time for release, some predatory mammals tend to rip, shred or chew their prey before swallowing the pieces. This action could destroy or damage the enteric coating of the pill resulting in poisoning of the predatory mammal.

The present disclosure overcomes the disadvantages of presently available methods to deal with invasive species. Accordingly, it is a general object of this disclosure to provide a targeted chemical delivery capsule.

It is another general object of the present disclosure to provide an improvement to ingestible drug delivery systems that allows targeted delivery of a drug or chemical to specific animals based on the ingestion method and digestive rate of the target animal.

It is a more specific object of the present disclosure to provide a selective drug delivery to animals with long digestive times while passing through animals with shorter digestive times without dispensing the drug.

It is another object of the present disclosure to provide delivery of the drug via live prey animals.

It is still another object of the present disclosure to provide a poison delivery package that can be implanted into a living prey animal.

It is a more specific object of the present disclosure to prevent premature delivery of the drug due to tearing, shredding or chewing action of the predator animals.

It is still another more specific object of the present disclosure to provide a mechanical means to prevent ingestion of the poison by desirable species.

These and other objects, features and advantages of this disclosure will be clearly understood through a consideration of the following detailed description.

### SUMMARY OF THE INVENTION

According to an embodiment of the present disclosure, there is provided a chemical delivery capsule assembly including a drug for poisoning a target species wherein the drug has an enteric coating for preventing dispensing of the drug in the stomach of an animal and a protective shell for preventing the premature puncturing of the coating by an animal.

According to an embodiment of the present disclosure there is also provided a system for passing a chemical through a predator mammal and targeting a predator reptile wherein a drug has an enteric coating with a thickness that does not break down while in the mammal digestion cycle but does while in a reptile digestion cycle thereby releasing the drug into the reptile.

According to an embodiment of the present disclosure there is also provided a method for targeting a chemical delivery to a specific species consisting of providing a drug capsule having an enteric coating, inserting the capsule into a bait; releasing the bait, and poisoning the specific species after the coating breaks down and releases the drug into the species.

### BRIEF DESCRIPTION OF THE DRAWINGS

The present disclosure will be more fully understood by reference to the following detailed description of one or more preferred embodiments when read in conjunction with the accompanying drawings, in which like reference characters refer to like parts throughout the views and in which:

FIG. 1 is an exploded front perspective view of the targeted chemical delivery capsule assembly according to the principles of an embodiment of the present disclosure.

FIG. 2 is a front perspective cutaway view of the assembly of FIG. 1 in an assembled state.

FIG. 3 is a side view of the assembly of FIG. 1.

FIG. 4 is an end view of the assembly of FIG. 1.

FIG. 5 is a cross-sectional view of the assembly taken along lines F5-F5 of FIG. 3.

FIG. 6 is a front perspective hidden line view of an assembly with an enteric coated capsule.

FIG. 7 is a front perspective view in an exploded state of the assembly of FIG. 6.

FIG. 8 is a front perspective cutaway view of the assembly of FIG. 6.

FIG. 9 is a side view of the assembly of FIG. 6.

FIG. 10 is an end view of the assembly of FIG. 9.

FIG. 11 is a cross-sectional side view taken along the lines F11-F11 of FIG. 9.

FIG. 12 is a front perspective cutaway view of an alternate embodiment of the present disclosure.

FIG. 13 is a front perspective view in an exploded state of the assembly of FIG. 12.

FIG. 14 is a side view of the assembly of FIG. 12.

FIG. 15 is an end view of the assembly of FIG. 14.

FIG. 16 is a cross-sectional side view taken along the lines F16-F16 of FIG. 14.

FIG. 17 is a front perspective cutaway view of an alternate embodiment of the present disclosure.

FIG. 18 is a front perspective view in an exploded state of the assembly of FIG. 17.

FIG. 19 is a side view of the assembly of FIG. 17.

FIG. 20 is an end view of the assembly of FIG. 19.

FIG. 21 is a cross-sectional view taken along lines F21-F21 of FIG. 19.

FIG. 22 is a front perspective view of an alternate embodiment of the present disclosure.

FIG. 23 is a front perspective view in an exploded state of the assembly of FIG. 22.

FIG. 24 is a side view of the assembly of FIG. 22.

FIG. 25 is an end view of the assembly of FIG. 24.

FIG. 26 is cross-sectional side view taken along lines F26-F26 of FIG. 24.

FIG. 27 is a front perspective view of an alternate embodiment of the present disclosure.

FIG. 28 is a front perspective view in an exploded state of the assembly of FIG. 27.

FIG. 29 is a side view of the assembly of FIG. 27.

FIG. 30 is a cross-sectional view taken along lines F30-F30 of FIG. 29.

FIG. 31 is a cross-sectional view taken along lines F31-F31 of FIG. 29.

FIG. 32 is a front perspective view of an alternate embodiment of the present disclosure.

FIG. 33 is a front perspective view in an exploded state of the assembly of FIG. 32.

FIG. 34 is a side view of the assembly of FIG. 32.

FIG. 35 is a cross-sectional view taken along lines F35-F35 of FIG. 34.

FIG. 36 is a front perspective view of an alternate embodiment of the present disclosure.

FIG. 37 is a front cutaway perspective view in an exploded state of the assembly of FIG. 36.

FIG. 38 is a side view of the assembly of FIG. 36.

FIG. 39 is a cross-sectional view taken along lines F39-F39 of FIG. 38.

FIG. 40 is a front perspective view of an alternate embodiment of the present disclosure.

FIG. 41 is a front perspective view in an exploded state of the assembly of FIG. 40.

FIG. 42 is a side view of the assembly of FIG. 40.

FIG. 43 is a cross-sectional view taken along lines F43-F43 of FIG. 42.

FIG. 44 is front perspective view of an alternate embodiment of the present disclosure.

FIG. 45 is a front perspective view in an exploded state of the assembly of FIG. 44.

FIG. 46 is a side view of the assembly of FIG. 44.

FIG. 47 is a cross-sectional view taken along lines F47-F47 of FIG. 46.

FIG. 48 is a side view of an embodiment similar to FIG. 44 with an added external encapsulation.

FIG. 49 is a cross-sectional view taken along lines F49-F49 of FIG. 48.

FIG. 50 is a front perspective view of an alternate embodiment of the present disclosure.

FIG. 51 is a front perspective view in an exploded state of the assembly of FIG. 50.

FIG. 52 is a side view of the assembly of FIG. 50.

FIG. 53 is a cross-sectional view taken along lines F53-F53 of FIG. 52.

FIG. 54 is a cross-sectional view taken along lines F54-F54 of FIG. 52.

FIG. 55 is a side view of an embodiment similar to FIG. 50 wherein the two shells are assembled.

FIG. 56 is a cross-sectional view taken along lines F56-F56 of FIG. 55.

FIG. 57 is a front perspective view of an alternate embodiment of the present disclosure.

FIG. 58 is a front perspective view in an exploded state of the assembly of FIG. 57.

FIG. 59 is a side view of the assembly of FIG. 57.

FIG. 60 is an end view of the assembly of FIG. 59.

FIG. 61 is a cross-sectional view taken along lines F61-F61 of FIG. 59.

FIG. 62 is a frontal perspective view of an alternate embodiment of the present disclosure wherein the chemical holding area is a hollow center created by intersecting cross drilled holes.

FIG. 63 is a front view of the assembly of FIG. 62.

FIG. 64 is a top plan view of the assembly of FIG. 63.

FIG. 65 is a side view of the assembly of FIG. 63.

FIG. 66 is a front perspective view of an alternate embodiment created by the addition of an encapsulating coating.

FIG. 67 is a front view of the assembly of FIG. 66.

FIG. 68 is a side view of the assembly of FIG. 66.

FIG. 69 is a cross-sectional view taken along lines F69-F69 of FIG. 67.

FIG. 70 is a front perspective view of an alternate embodiment of the present disclosure wherein the chemical holding area is a hollow center created by forming an outer skin around the hollow center.

FIG. 71 is a front view of the assembly of FIG. 70.

FIG. 72 is a top plan view of the assembly of FIG. 70.

FIG. 73 is a side view of the assembly of FIG. 70.

FIG. 74 is a cross-sectional view taken along lines F74-F74 of FIG. 73.

FIG. 75 is a front perspective view of an alternate embodiment created by the addition of an encapsulating coating.

FIG. 76 is a front view of the assembly of FIG. 75.

FIG. 77 is a cross-sectional view taken along lines F77-F77 of FIG. 76.

FIG. 78 is a cross-sectional view taken along lines F78-F78 of FIG. 76.

FIG. 79 is a front perspective view of an alternate embodiment of the present disclosure.

FIG. 80 is a cutaway exploded sectional front perspective view of the assembly of FIG. 79.

FIG. 81 is a side view of the embodiment of FIG. 79.

FIG. 82 is a side sectional view taken along lines F82-F82 of FIG. 81.

FIG. 83 is a profile view of a prey animal with a capsule of the present disclosure inserted subcutaneously.

FIG. 84 is a frontal perspective view of an alternate embodiment of the present disclosure wherein a spring is used as the shell to resist the shredding and tearing of the enclosed cargo of the enteric coated capsule.

FIG. 85 is a cutaway front perspective view of the assembly of FIG. 84.

FIG. 86 is a plan view of the assembly of FIG. 84.

FIG. 87 is an end view of the assembly of FIG. 86.

FIG. 88 is a cross-sectional side view taken along lines F88-F88 of FIG. 86.

FIG. 89 is a plan view of the assembly of FIG. 84 with an added cord.

FIG. 90 is cross-sectional frontal view taken along lines F90-F90 of FIG. 89.

FIG. 91 is a frontal profile view of the outline of a prey animal which the assembly of FIG. 90 has been implanted according to the principles of an embodiment of the present disclosure.

FIG. 92 is a frontal perspective view of a flat thin capsule embodiment according to the principles of the present disclosure.

FIG. 93 is a cutaway front perspective view of the assembly of FIG. 92.

FIG. 94 is a plan view of the assembly of FIG. 92.

FIG. 95 is cross-sectional side view taken along lines F95-F95 of FIG. 94.

FIG. 96 is an end view of the assembly of FIG. 94.

FIG. 97 is a frontal perspective view of an alternate embodiment of a flat thin shaped capsule under a patch according to the principles of the present disclosure.

FIG. 98 is frontal perspective view of the patch exploded away from the underlying capsule of the assembly of FIG. 97.

FIG. 99 is a plan view of the assembly of FIG. 97.

FIG. 100 is a cross-sectional side view taken along lines F100-F100 of FIG. 99.

FIG. 101 is a cross-sectional end view taken along lines F101-F101 of FIG. 99.

FIG. 102 is a frontal profile view of the outline of a prey animal in which a flat pack embodiment of FIG. 97 has been attached according to the principles of the present disclosure.

FIG. 103 is a cutaway frontal perspective view of an alternate embodiment capsule utilizing a shell rolled around an enteric coated pill according to the principles of the present disclosure.

FIG. 104 is an exploded frontal perspective view of the assembly of FIG. 103.

FIG. 105 is a plan view of the assembly of FIG. 103.

FIG. 106 is a cross-sectional view taken along lines F106-F106 of FIG. 105.

FIG. 107 is a cross-sectional view taken along lines F107-F107 of FIG. 106.

FIG. 108 is a frontal perspective view of a capsule of an embodiment of the present disclosure in which the chemical cargo and the enteric coating are applied to the shell material prior to rolling the shell into final shape.

FIG. 109 is a cutaway frontal perspective view of the assembly of FIG. 108.

FIG. 110 is a plan view of the assembly of FIG. 108.

FIG. 111 is a cross-sectional view taken along lines F111-F111 of FIG. 110.

FIG. 112 is a cross-sectional view taken along lines F112-F112 of FIG. 110.

FIG. 113 is a frontal perspective view of the components of the capsule of FIG. 108 prior to being rolled into final shape.

FIG. 114 is a plan view of the assembly of FIG. 113.

FIG. 115 is a cross-sectional view taken along lines F115-F115 of FIG. 114.

FIG. 116 is an enlarged detailed view of the F116 section of FIG. 115.

### DESCRIPTION OF THE PREFERRED EMBODIMENTS

The invention is an ingestible chemical delivery system for delivery of drugs, poisons, or other chemicals to a targeted predator animal via a prey animal while preventing delivery to mammal species.

The disclosure consists of a pill or capsule containing the desired drug which can be covered in an enteric coating to prevent the stomach acids from dissolving or interacting with the drug before it reaches the intestine. The disclosure also includes a chew proof shell element that protects the pill and enteric coating from being broken up and dispensed prematurely by tearing, shredding, or chewing action of the consuming animal on the prey or bait. The invention may also include an outer encapsulation coating that allows the capsule to survive in the subcutaneous fat layer of a prey animal layer without the drug being dispensed into the prey animal.

One or more embodiments of the subject disclosure will now be described with the aid of numerous drawings. Unless otherwise indicated, use of specific terms will be understood to include multiple versions and forms thereof. Examples of such versions include, but are not limited to the terms below:

CARGO is the chemical or drug that is desired to be delivered. It can be assembled into multi part capsule, pressed pill, bag, or other container capable of holding the chemical, drug, or poison. The CARGO can be in any form including solid, granulated, powdered, or liquid.

SHELL encases at least a portion of the pill to prevent the enteric coating from being damaged by ripping, shredding, or chewing action of a predator animal.

ENTERIC COATINGS are used to prevent stomach acids from dissolving the pill and dispensing the drug in the stomach while allowing the pill to move on to the intestine intact where it can dissolve in the more neutral PH of the intestine and dispense the chemical, drug, or poison.

ENCAPSULATION is an outer coating, skin, or membrane that will not dissolve in the subcutaneous fat layer of a prey animal that may or may not add more time delay prior to the dissolving of the desired drug or chemical.

CUPPED SHELL has one at least partially closed end and one open end.

PLUG may be used to contain the CARGO in the SHELL and may add more time delay prior to the dissolving of the desired drug or chemical.

TRACER can be a metallic, radioactive, or other material that can be detected with metal detectors, x-ray, sonogram, or other imaging equipment. This can also be a radio transmitter or, instrument, or a data logger.

RIMs inserted into the open end of a SHELL can strengthen the open end against crushing forces.

SPHERICAL SHELL is a hollow spherical shape can be used to hold powdered, granulated, or liquid CARGO.

CROSS DRILLED HOLES is when a SHELL constructed by creating a hollow in a portion of a solid material with intersecting cross drilled holes.

PREY ANIMAL is the animal that the components of the Targeted Chemical Delivery Capsule is implanted into.

PREDATOR BAIT is the portion of bait that the components of the Targeted Chemical Delivery Capsule is implanted into.

ENTERIC FILLING is a filling that can be placed in the shell with the cargo or pill to give added enteric protection time delay.

CAPSULE is the assembly of the components described in the present disclosure that will allow achievement of the objects of the present disclosure.

While this disclosure may focus on a solution to specific invasive species, such as the Burmese Python, it will be appreciated that it can be utilized for other species, including but not limited to feral pigs, non-native iguanas, etc. Indeed, while multiple embodiments are intended to be implanted into prey animals that will be captured alive and eaten by the intended target predator, the capsule can also be inserted into plants such as fruits and/or vegetables that the intended target animal may eat. For example, it is common Florida practice to lace strawberries with acetaminophen powder to kill iguanas and other lizards.

The disclosure and its embodiments within works by being first implanted in a number of prey animals of the target predator animal or in pieces of bait that the target predator may eat. Implantation location may be under the skin in the subcutaneous fat layer, in the abdominal cavity, or other convenient location of a prey animal, or simply inserted into a piece of bait. As an alternate to implantation the prey animal may be banded with a banding device such as a bird band or a fish tag. A number of these prey animals are then released into the environment of the targeted predator animal, which allows the predator animals to catch and eat some of the prey animals.

Initial Ingestion. In the case of the desired target predator of a snake, the snake eats the prey whole without chewing and the prey with the implanted device passes to the stomach of the snake with the components mostly intact.

In the case of a carnivore, such as a canine or a feline, the predator may rip, shred, and chew the flesh of the prey animal. The chew proof shell prevents the drug containing capsule component from being penetrated and broken up and thereby prevents premature release of the drug into the predator.

Stomach. Upon entry to the stomach the outside encapsulation of the pill begins to be dissolved by the stomach acid. With a long enough residence time in the stomach the stomach acid next dissolves the chew proof shell in the case of an acid sensitive metallic shell. In an alternate construction the chew proof Shell is perforated and allows the stomach acid to penetrate the shell to gain access to the enteric coating of the Capsule containing the drug. The enteric coating does not entirely dissolve in the acid, low PH, environment of the stomach.

Intestine. Upon entering the intestine, the enteric coating is exposed to the neutral to basic PH of the small intestine where it begins to dissolve. By making this enteric coating thick enough it can pass thorough a feline or canine intestine before the drug in the pill is exposed, thereby passing harmlessly through the digestive tract of a mammal and exiting in the feces. Since the time of the digestive cycle of a large snake is much longer than that of a mammal the same enteric coating will be dissolved while still in the intestine of a large snake, thereby delivering the drug dosage deep in the intestine of the snake.

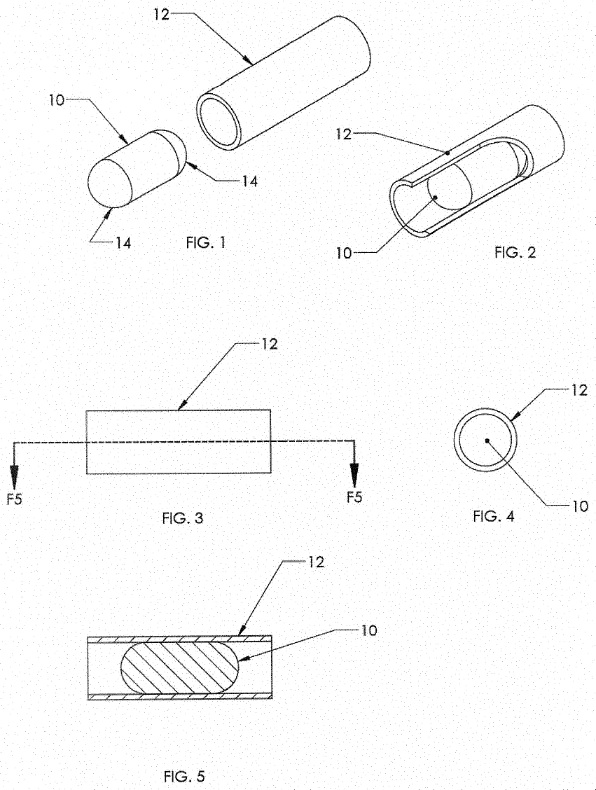

Turning now to the Figures, FIG. 1 shows a basic embodiment of the present disclosure wherein a pill capsule 10 is pressed into a tubular shell 12. The shell provides resistance to crushing forces from chewing, shredding, or ripping by predator animals. A plan view of this embodiment is shown in FIG. 3. The pill inside the shell can be seen in FIG. 4. FIG. 5 shows a cross section of this embodiment. This embodiment can be used for chemical or drug material that will not dissolve in the stomach acid of an animal but will start to dissolve in the more neutral PH of the intestines. Since only the ends 14 of the pill are exposed to the intestinal fluids the pill 10 will dissolve more slowly than if it did not reside on the tubular shell 12. For some chemicals this will be enough not to deliver a lethal dose to a mammal predator with a short digestive cycle time, but the much longer digestive cycle time of a large reptile will dissolve much more of the cargo chemical.

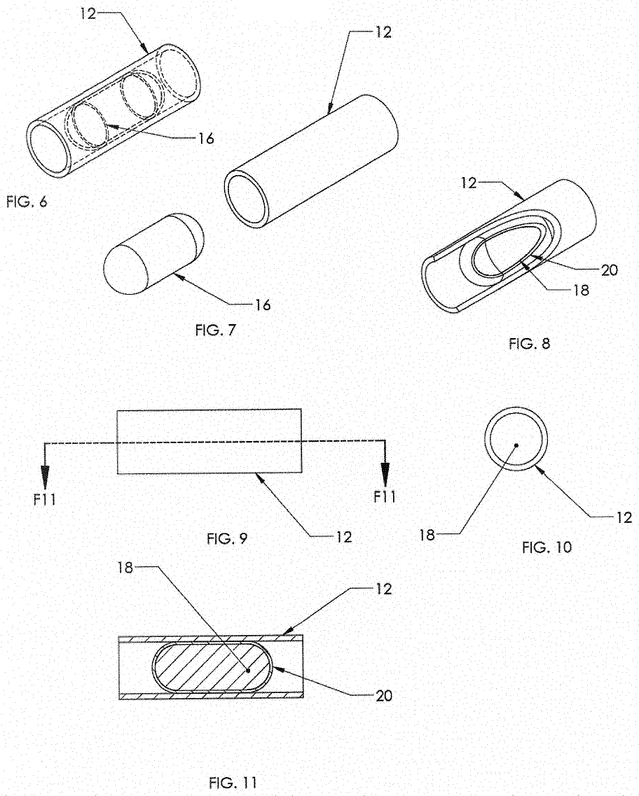

FIGS. 7 through 11 show a similar embodiment wherein the cargo drug has an enteric coating. FIGS. 6 and 7 show an enteric coated pill 16. FIG. 8 shows the shell 12 and the cargo 18 and enteric coating 20 components of the enteric coated pill 16. FIG. 11 is a section view that shows the cargo 18, shell 12, and this enteric coating 20.

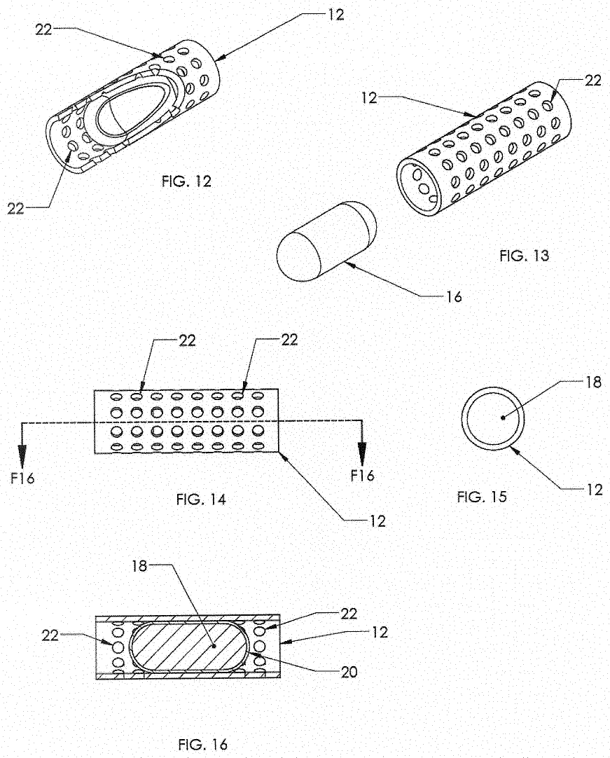

FIGS. 12 through 16 show a variation wherein the shell 12 is perforated 22 to allow the intestinal fluids more access to a basic pill or the enteric coated pill 16 shown. FIG. 16 is a section view showing the details of a shell 12 in perforated tube form, the cargo 18, and the enteric coating 20.

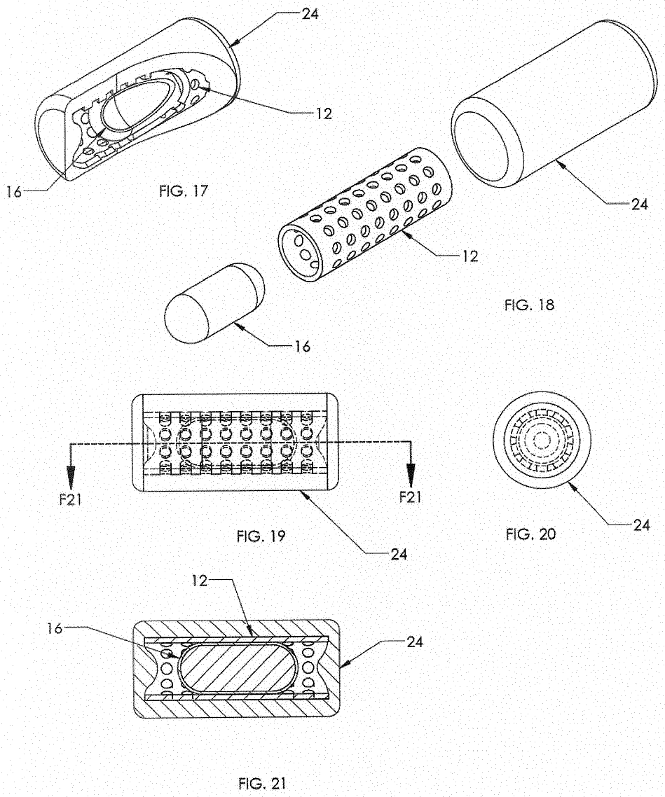

FIGS. 17 through 21 show the embodiment of FIG. 12 covered with an encapsulation 24. Since one intended usage of the present disclosure is for it to be implanted into the sub-cutaneous fat layer of a prey animal this encapsulation 24 may create a smoother package that can be implanted easier and be of a material that will not react with the fat layer of the prey animal. In addition, this encapsulation can serve as an enhancement or replacement for the enteric coating of a basic pill to insure that the encapsulated pill passes through a mammal but still will dissolve in the digestive system of a large reptile.

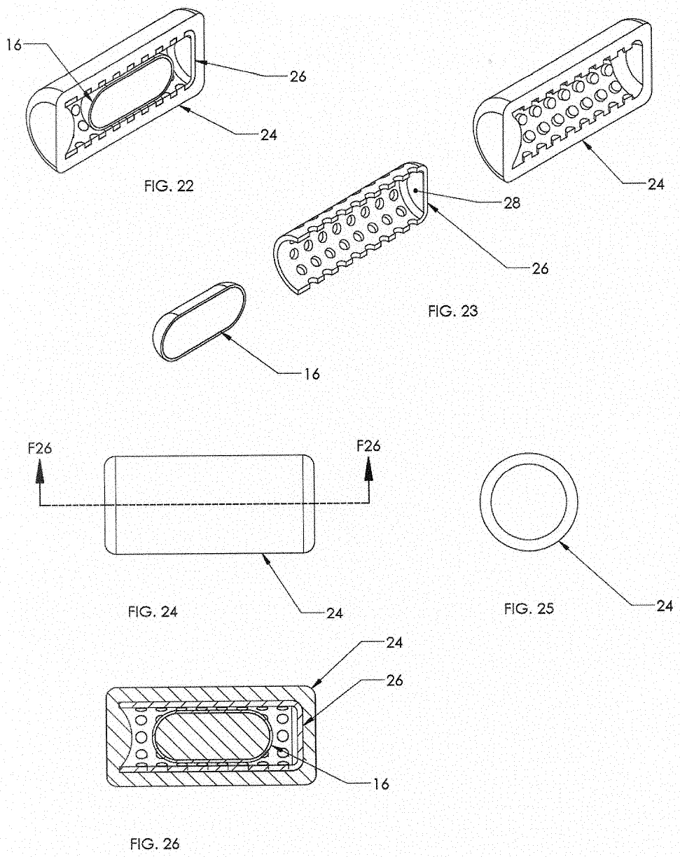

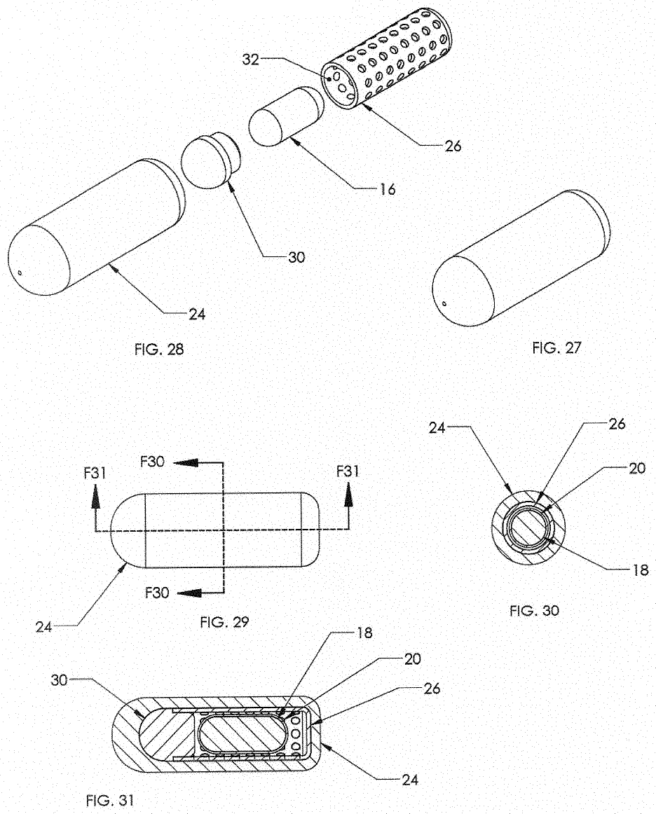

FIGS. 22 through 26 show a cupped shell 26. The closed end 28 of the cupped shell 26 makes it much more resistant to crushing forces than a simple tube of the same thickness. The cupped shell 26 is also more convenient to assemble various size pills into since they do not have to be a friction fit with the tubular shell walls.

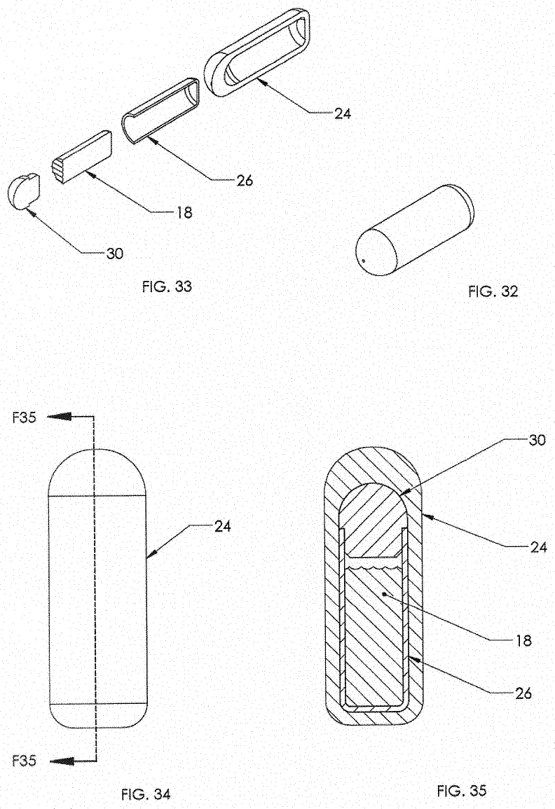

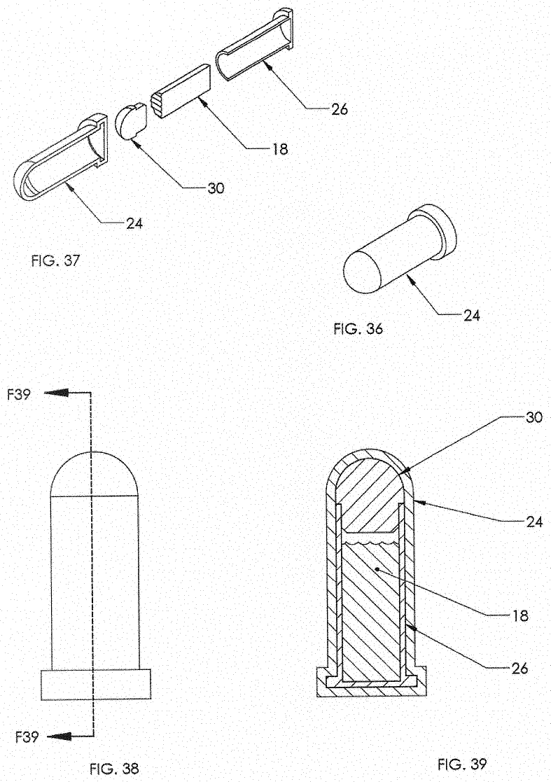

FIGS. 28 through 31 show a cupped shell 26 with an enteric coated pill 16, the encapsulation 24, with a plug 30 added to the assembly. This plug 30 can serve multiple purposes. One purpose is to make the open end 32 of the cupped shell 26 stronger and more resistant to crushing forces. The plug can also serve to delay dissolving of the cargo 18 chemical. The material of the encapsulation 24 may be chosen to dissolve rather quickly while the material of the plug 30 can be chosen to be much more resistant to dissolving in intestinal fluids. If the cupped shell 26 is made without the perforations shown in FIGS. 28-31 the plug 30 will be the major factor controlling the final dissolving of the enteric coating 20 and the cargo 18.

FIGS. 33 through 35 show a cupped shell 26 without perforations, a plug 30, an encapsulation 24, and a liquid cargo 18. The cupped shell 26 without perforations allows delivery of a chemical 18 in any form including powders, solids, granulations, and liquids. This embodiment can be used with or without the shown encapsulation 24.

FIGS. 36 through 39 show a cupped shell 26 in the form of a brass rifle cartridge, a plug 30, an encapsulation 24, and a liquid cargo 18. The commonly available brass shell also allows delivery of a chemical in any form including powders, solids, granulations, and liquids. Since the brass rifle cartridge is so commonly available it presents a low-cost production startup option of purchasing the rifle cartridges instead of making cupped shells. This embodiment can be used with or with the show encapsulation 24. The brass of the rifle cartridge is resistant to dissolving in low PH acids of the stomach and the neutral to basic PH of the intestinal fluids, so it has the added benefit of being able to be detected by metal detectors, X-rays, and sonograms if researchers want to know where in the digestive system the cartridge was when the reptile died from the specific cargo dose.

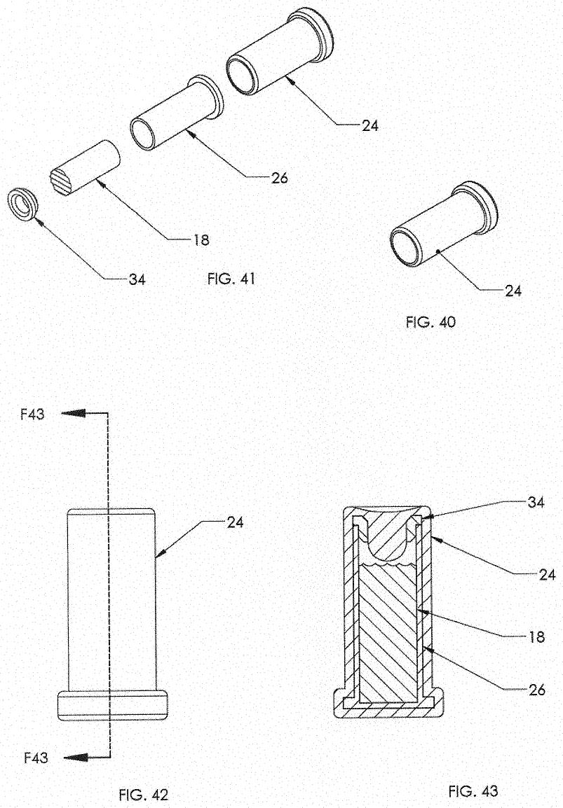

FIGS. 40 through 43 show a rifle cartridge shaped embodiment that uses a reinforcing rim 34 instead of a plug. It may be desirable to not use a solid plug if it delays the dissolving of the chemical cargo for too long. However, the open end of the cupped shell 26 may not be strong enough to stand up to the crushing force of predator animals chewing, shredding, and ripping the prey animal. In this case an annular ring-shaped rim 34 can be inserted in the open end of the cupped shell 26. The opening size in this rim 34 can be tailored to allow the desired rate of bodily fluid access to the cargo chemical 18. These figures show a liquid cargo 18 and an encapsulation 24 but it will be understood that any form of cargo chemical can be used with this embodiment and that the encapsulation is optional.

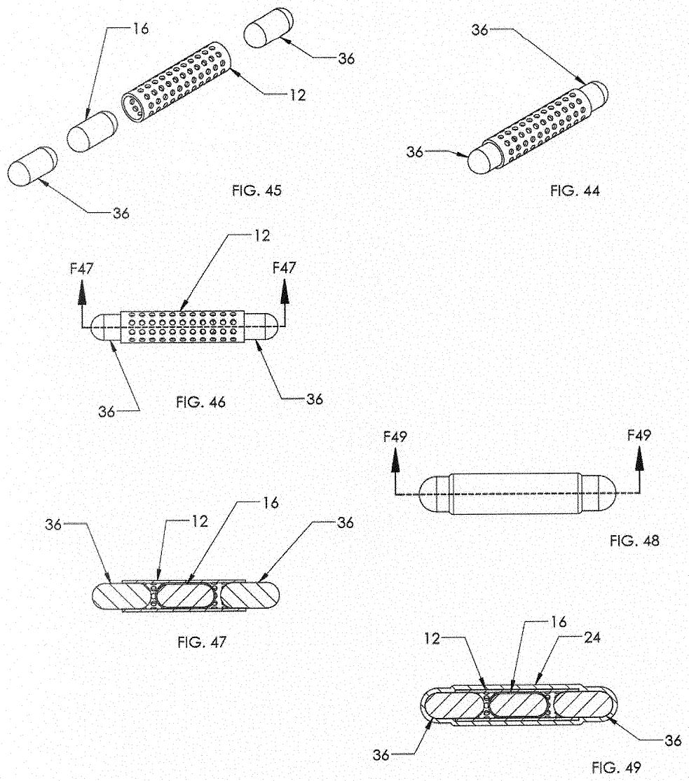

FIGS. 44 through 49 show an embodiment wherein a simple tube is used as the shell 12 and tracers 36 are used to enable tracking of the capsule through an animal's body, to locate the animal in the wild or to collect data. As shown in these figures the enteric coated pill 16 is inserted in the shell 12 and the pill is captured in the tube by the tracers 36. If the tracers are made from copper, silver, nickel, or other acid resistant metals the tracers can be used a tracer that will be detectable by metal detectors, x-rays, sonograms, and other imaging technologies. The strength of these materials will make the assembled cartridge stronger to better resist the chewing, shredding, and ripping forces of predator animals. Notice that encapsulated and non-encapsulated version of this embodiment are shown. It will be understood the tracers can be used with other embodiments in this disclosure and that any number of tracers can be used.

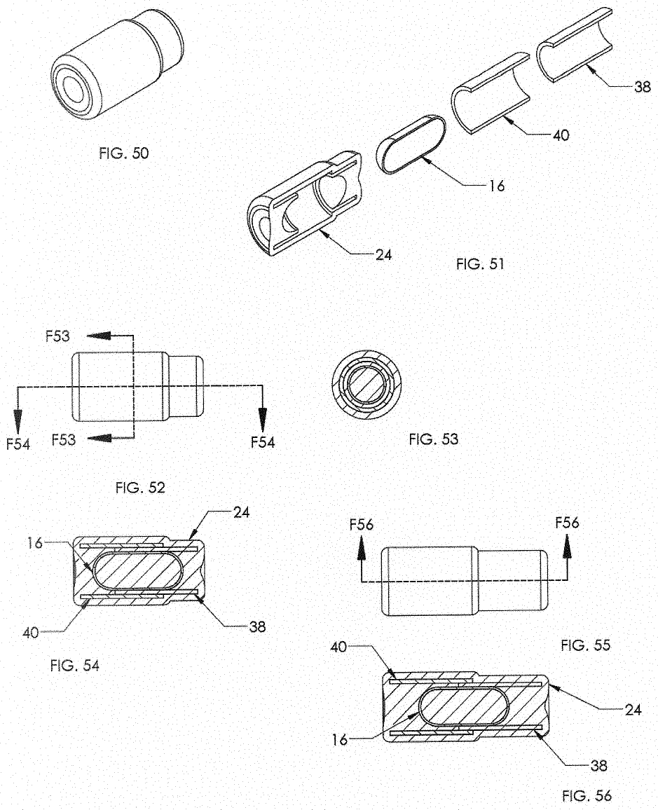

The disclosure up to this point will allow the construction of a targeted drug delivery capsule that can be custom made for delivery into the digestive tract of a specific predator reptile. However, a system based on this concept would be much more flexible if the size of the capsule could be adjusted without the need for different parts. FIGS. 50 through 56 show an embodiment using two tubular elements 38, 40 as the shell. By assembling these two tubes 38, 40 to different heights different length capsules can be made. These resulting different length shell assemblies can hold different size enteric coated pills 16 or they can hold different amounts of encapsulation material to delay release of the cargo drug. It is understood that the two referenced shells can be in any form including cupped shells, tubes, irregular shapes or any combination of these forms.

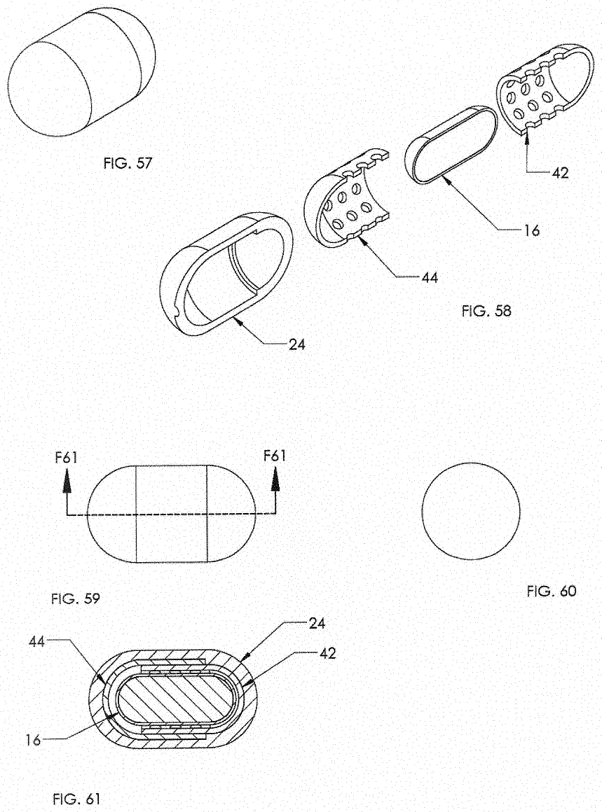

FIGS. 57 through 61 show an embodiment using two perforated capsule halves 42, 44 as the shells. The previous discussion applies to this embodiment with the exception that it combines the concept of a cupped shell with the slightly different size shells to allow the shell halves 42, 44 to overlap each other during assembly thereby creating a degree of variability in final assembly size.

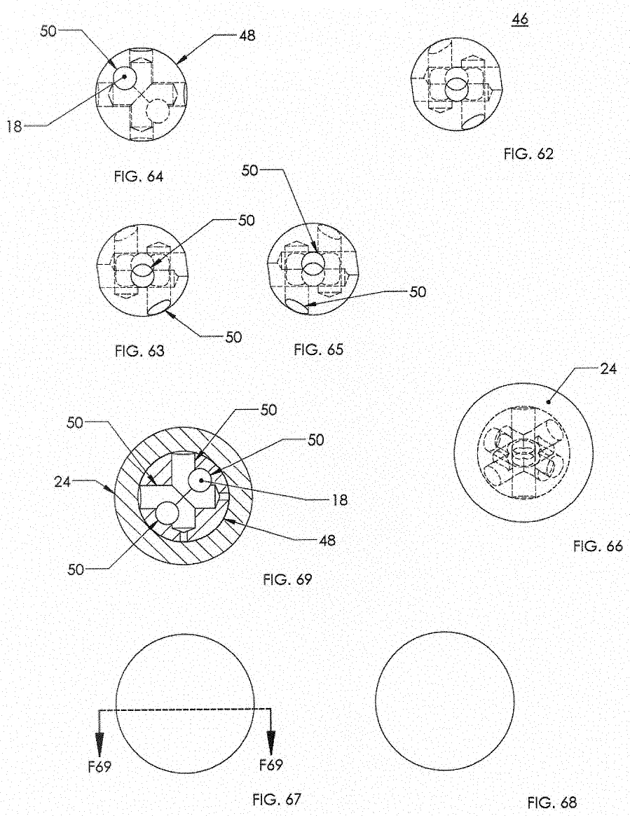

It is possible to implement this disclosure in an endless array of shapes. One possible shape is a hollow ball shape 46 shown in FIGS. 62 through 68. FIG. 62 shows a spherical shell 48 that is given a hollow interior area by adding in cross drilled holes 50. The resulting hollow volume is then filled with the cargo 18 chemical. Once filled, the filled assembly is coated with an enteric coating and then an encapsulation 24 coating. FIGS. 62 through 65 show the basic cross drilled spherical shell with the cargo 18 chemical packed into the hollow volume. FIGS. 66 through 69 show this shape after an encapsulation 24 has been added. This shape has a structural advantage over the tube-based concepts when it comes to resisting crushing forces from predator animals chewing, shredding, and ripping the prey or bait.

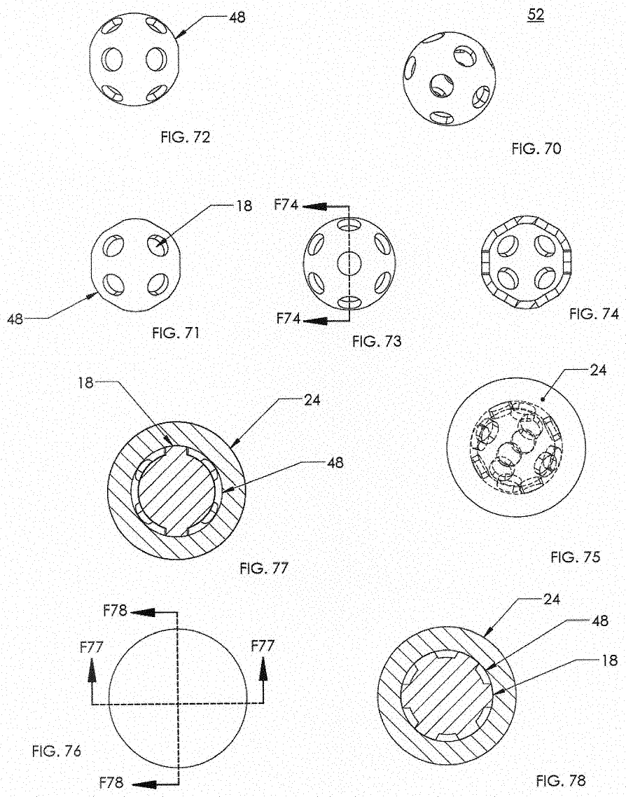

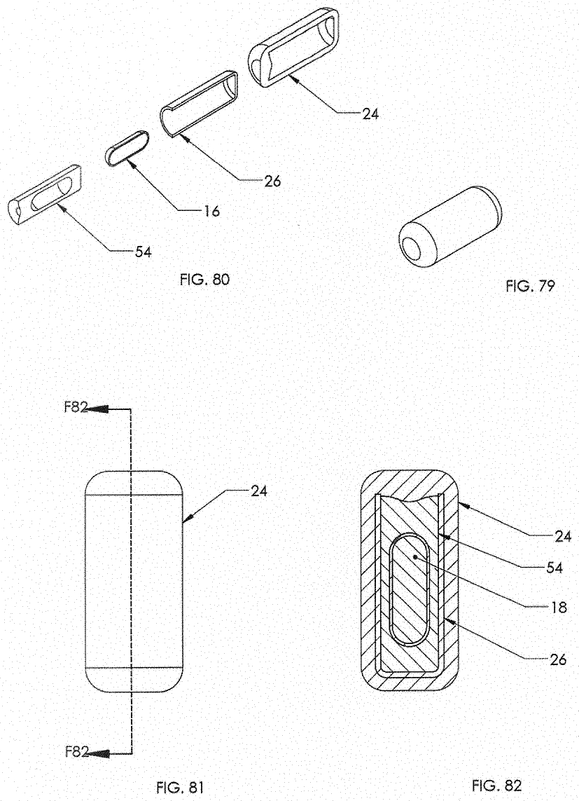

At higher production volumes a cast or formed spherical shell would be more cost effective than the cross drilled holes show in FIGS. 62 through 69. FIGS. 70 through 78 show a hollow cast ball-based 52 embodiment. FIGS. 70 through 74 show a cast ball with cast in holes as the spherical shell 48. FIGS. 75 through 78 show this spherical shell with an encapsulation added. FIGS. 77 and 78 show section views through the capsule to show the cargo 18 chemical, the encapsulation 24 thickness, and the walls of the spherical shell 48.

Another embodiment of the present disclosure places an enteric filler into the shell with a pill to give added time delay. The enteric filler can be seen as item 54 in FIGS. 80 and 82.

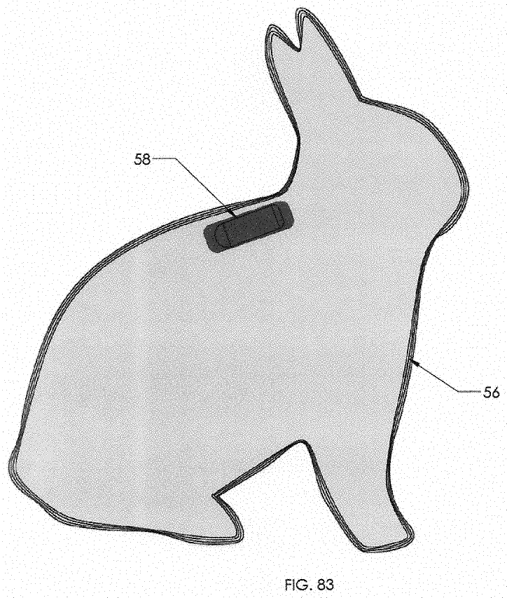

While it is possible to insert the above embodiments into the throat of a captive snake or large reptile the animal will probably not voluntarily swallow one of the capsules. Since it is known that large constrictor type snakes prefer to only eat prey that they have killed themselves it is desirable to first place this targeted chemical delivery capsule into a number of prey animals that the target predator reptile normally eats. FIG. 83 shows a profile outline of a rabbit 56 which is a normal prey animal of large snakes. The targeted chemical delivery cartridge 58 is shown in schematic form after being inserted under the skin of the prey animal.

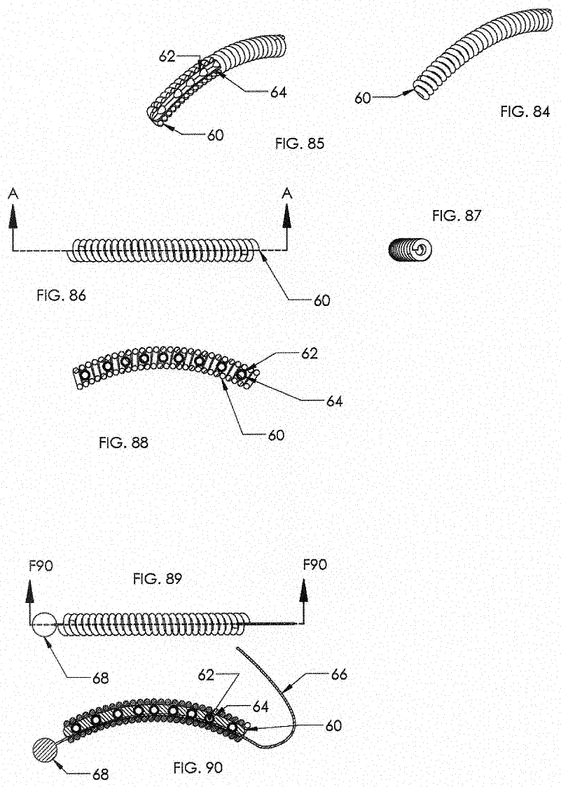

FIGS. 84 through 88 show an embodiment wherein a simple spring shell 60 is used to create a long thin flexible shape that may be easier to implant into a prey animal, animal part bait, or fruit or vegetable bait. FIG. 85 shows round cargo carrying pills 62 contained in a flexible spring shell 60 which is then filled with enteric material 64 to delay dispensing of the chemical cargo until the desired time after ingestion by the target animal. As shown in FIG. 88 the digestion delaying function of the enteric coating can be achieved by coating the individual cargo containing pills 62 thereby negating the need of filling the shell 60 element with a separate enteric material 64. It is understood that the target chemical carrying object can be in any form including absorbent sheets or shapes that have been previously loaded with the cargo chemical.

The implantation location of the invention may be under the skin in the subcutaneous fat layer, in the abdominal cavity, or other convenient location of a prey animal, or simply inserted into a piece of bait. Insertion of the invention into the prey animal or bait may be simplified by being able to thread the invention into position without the need for a surgical incision and the attendant dangers of infection. FIGS. 89 to 90 show an extension of the concept shown in FIGS. 84 to 88 to which a thread or cord 66 with a knot 68 or other protrusion has been added. This thread allows the use of a needle to pierce the skin of the prey animal or bait and then pull this long thin flexible version of the invention into position.

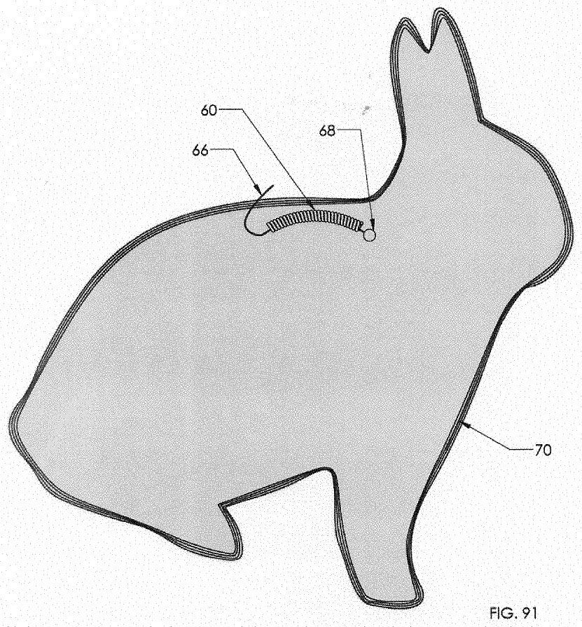

FIG. 91 shows a profile outline of a rabbit which is a normal prey animal 70 of large snakes. The spring shell embodiment 60 of the present disclosure is shown in schematic form after being inserted under the skin of the prey animal. The cord 66 is pulled under the skin of the prey animal using a needle while the knot 68 on the end of the cord prevents the cord from being pulled through the spring shell in the event that the spring shell forms a hollow tube. As an alternate installation method, a doubled cord may be uses to initially place the spring shell container in the desired location. The cord can then be removed by releasing one end of the cord and pulling on the other end to draw the cord through the wound cavity created by the needle and out of the prey animal. The knot function of the cord can be achieved by any protrusion of suitable size such as a crimped-on bead, a radio tracking device, or any device that can be attached to the end of a cord.

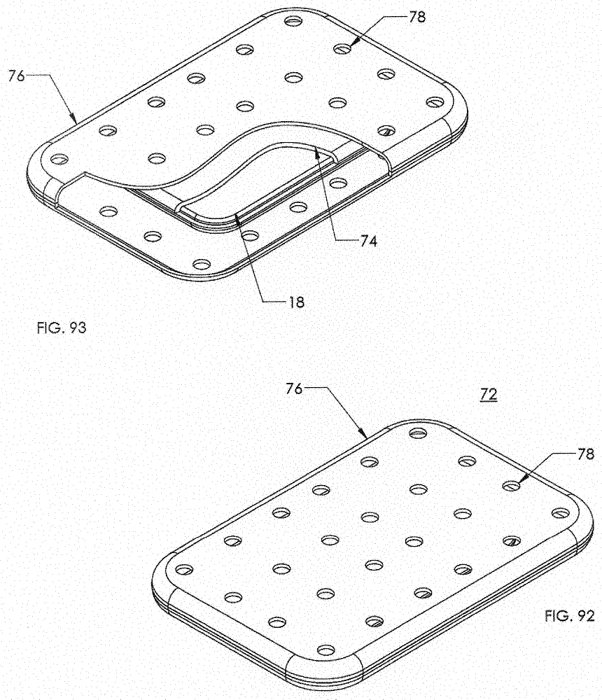

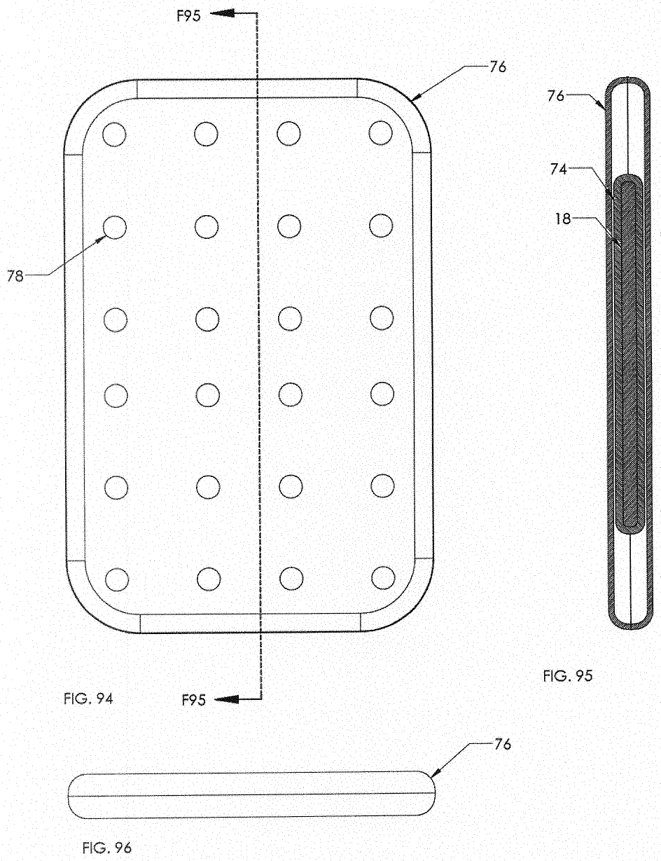

It may be desirable for the invention to not have the cylindrical shape shown elsewhere in this disclosure. FIG. 92 shows a basic embodiment of the present disclosure wherein a simple pill or capsule 72 is pressed into a flattened shape. The cargo 18 containing flattened shape is then encased in an enteric material 74. This enteric coated pill shape in then placed into a flattened shell 76 that is strong enough to resist the crushing forces from chewing, shredding, or ripping by desirable native predator animals. Holes or porosity 78 of the shell material allow eventual intrusion of intestinal fluids to dissolve the enteric coating and release the cargo chemical into the targeted predator animal. It is understood that like other embodiments in this disclosure this flat pack embodiment may be coated with a biologically friendlily material for compatibility with the bodily material of the prey animal.

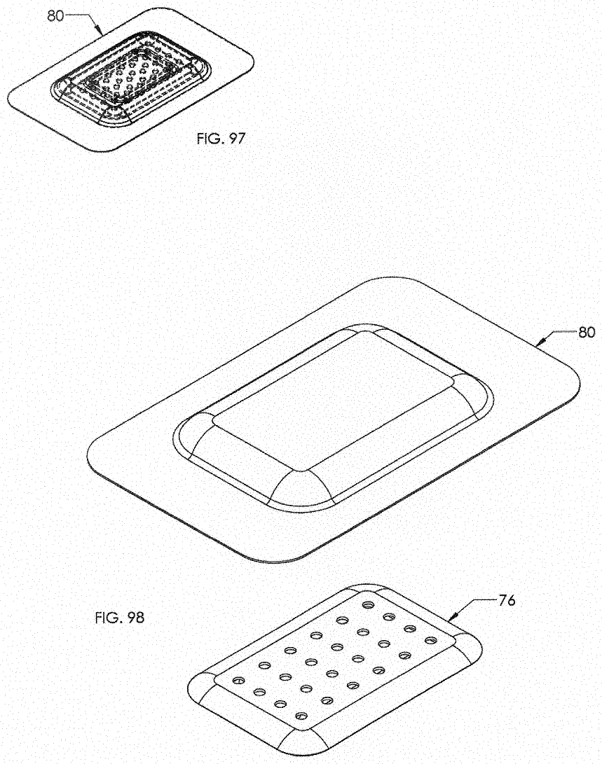

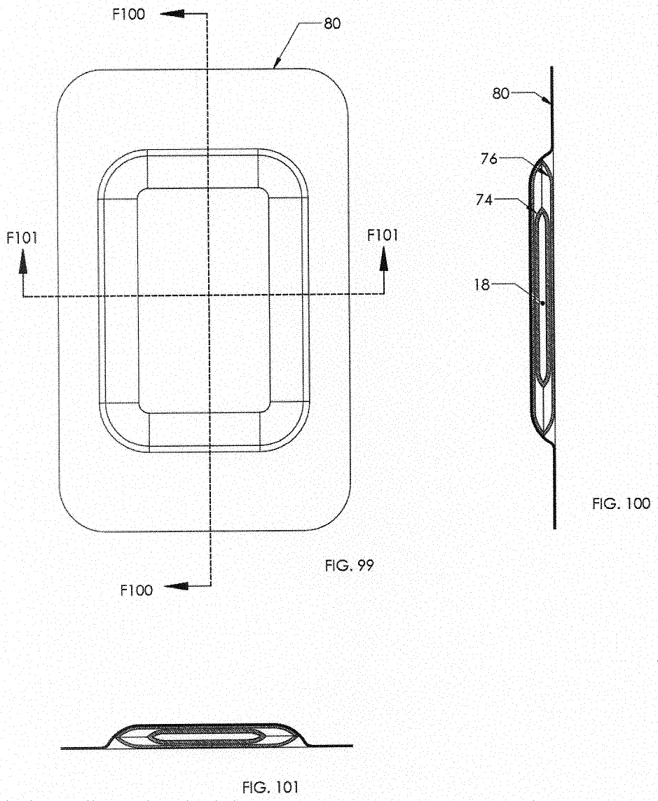

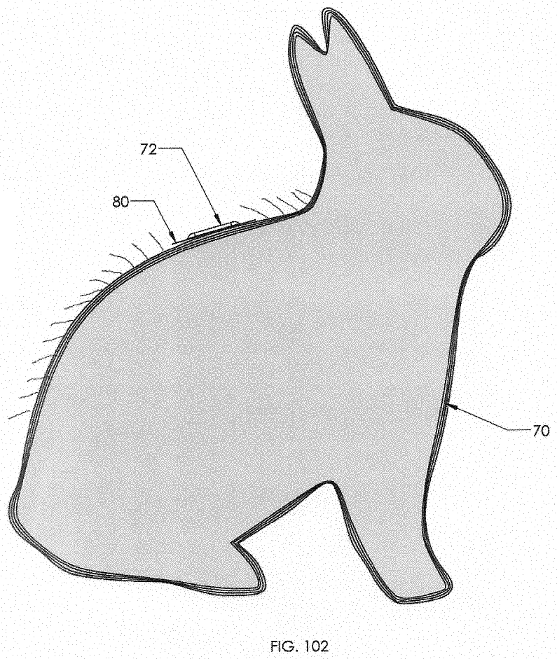

One advantage of the flat pack shown in FIGS. 97 to 101 is that it can be attached to exterior areas of the prey animals that are inaccessible to the prey animals scratching or chewing actions in attempts to remove the flat pack such as the center of their backs. This eliminates the need for veterinarians capable of performing invasive surgical operations into the bodies of prey animals with surgical or needle implants. A very simple mounting of this flat pack on a prey animal could be achieved by securing the flat pack version to a prey animal with adhesive or an adhesive patch or bandage 80. This would reduce the skill level required to install the invention from that of a basic surgeon or veterinarian to that of technician capable of simply shaving some hair from the body of the prey animal and attaching the invention with adhesive or an adhesive patch 80. FIG. 97 shows the adhesive patch 80 over the flat pack capsule 72 embodiment of the invention shown in FIG. 92. FIG. 98 shows an exploded view of this installation. FIG. 99 shows a plan view of this embodiment. FIGS. 100 to 101 show section views with the cargo 18, the enteric coating 74, the shell 76, and the adhesive patch 80.

FIG. 102 shows a profile outline of a rabbit 70 which is a normal prey animal of large snakes. The flat pack 72 embodiment of the container of the present disclosure is shown in schematic form after being attached to the skin of the prey animal 70 with an adhesive or adhesive patch 80. One method of achieving this attachment would be to shave the hair from a section of the skin of the prey animal, clean the skin to remove body oils, and apply the adhesive or adhesive patch with this flat pack embodiment of the container. The option to adhesively apply the present disclosure to a prey animal without the need for the skills of a veterinarian could greatly reduce the costs and time to implement this invention into an invasive non-native species situation.

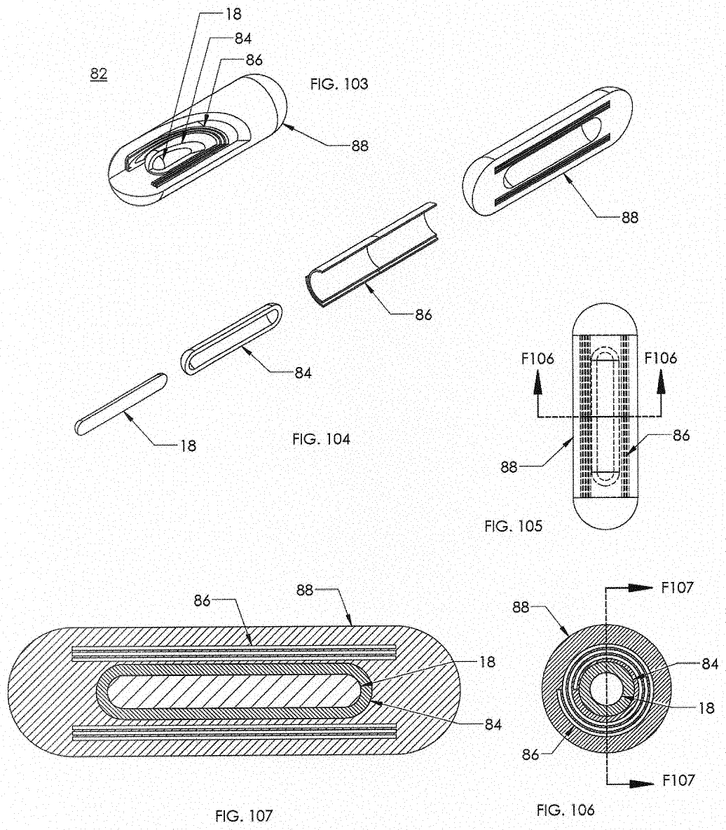

For ease of manufacture, it may be desirable to achieve the required level of resistance to the chewing, ripping and shredding action of the teeth of desirable native predator animals by forming the protective shells from a thin sheet or sheets of suitable material formed around an enteric coated cargo carrying pill. FIG. 103 shows a cylindrical shaped 82 version of the present disclosure where in the cargo 18 is encased in an enteric material 84 and is then rolled in a suitable number of layers 86 of material suitable for the shell. Once the cargo 18, the enteric coating 84, and the shell 86 are assembled they are encased in a biologically friendly coating 88. FIG. 104 shows an exploded view of FIG. 103. FIG. 105 shows a plan view of FIG. 103. FIG. 106 shows a sectional end view of FIG. 105 along lines F106-F106. The relationship of the components in this embodiment can be seen more clearly in FIG. 107 side sectional view of FIG. 106.

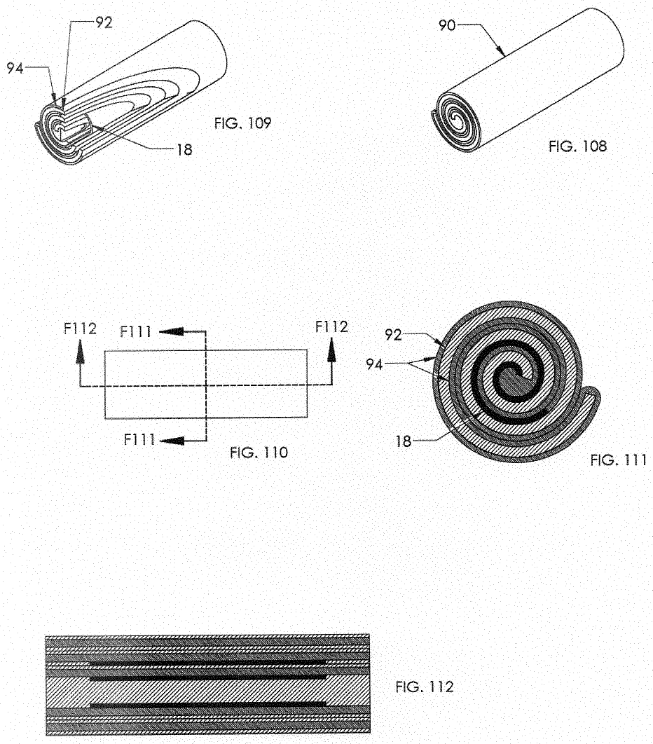

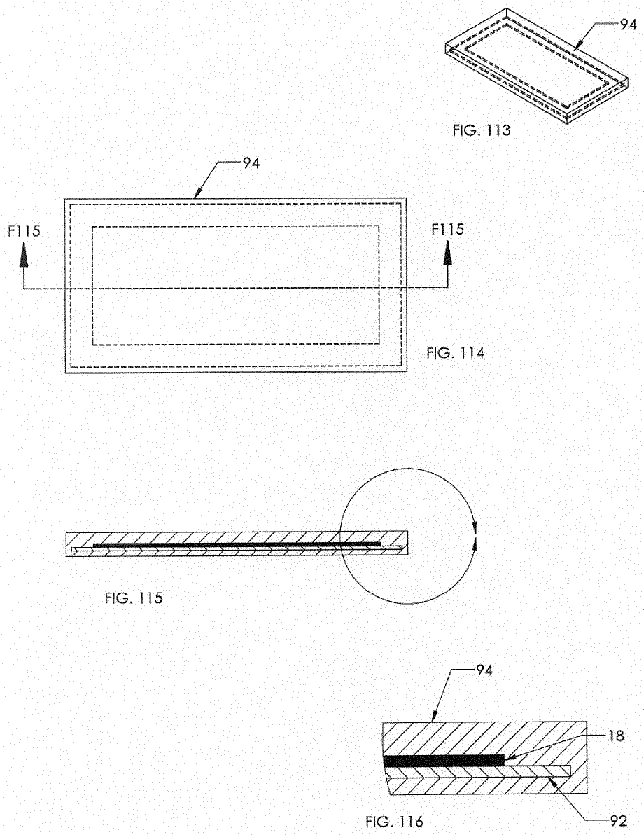

For additional ease of manufacture, it may be desirable to simply coat the shell material with the cargo chemical and the enteric coating prior to rolling them all into a cylindrical shape in one operation. FIG. 108 shows a rolled cylindrical shaped version 90 of the present disclosure wherein the shell material is coated with the cargo chemical and then both are coated with the enteric material. FIG. 109 shows a perspective cut away view that exposes the cargo 18 chemical layer, the enteric coating 94 layer, and the shell 92 layer. FIG. 110 is a plan view of FIG. 108. FIG. 111 shows an end sectional view along lines F111-F111 of FIG. 110 which exposes the different layers of the present disclosure. FIG. 112 is a side section view of FIG. 110 along lines F112-F112. FIGS. 111 to 112 highlight the fact that the cargo chemical is fully protected from contact with any early action by stomach acids by virtue of not extending the to the edges of the shell cylinder and being fully covered by the enteric coating.

FIG. 113 is a perspective view of the coated shell material prior to rolling into the container assembly. FIG. 114 shows a plan view of FIG. 113. FIG. 115 shows a section side view of FIG. 114 along lines F115-F115. FIG. 116 shows a detail view of FIG. 115 showing the relationship of the shell material 92, the chemical cargo 18, and the enteric coating 94. It is understood that the target chemical can be applied to the shell material by any process including brushing, dipping, or spraying or that the cargo chemical can be applied via the use of absorbent sheets or shapes that have been previously loaded with the cargo chemical.

As described herein, the ingestible targeted chemical delivery capsule can be built by starting with a pill or container that contains the desired chemical, drug, or poison. An enteric coating is then applied to the exterior of the pill. When it is intended to deliver the drug specifically to reptiles without drugging mammals this coating may be much thicker than the enteric coating on familiar human targeted drugs to delay the release of the drug long enough to pass through mammals and exit with the feces without dispensing the drug. This enteric pill is then placed in a protective shell that is strong enough and stiff enough to protect the pill and the enteric coating of the pill from damage by the ripping, shredding, or chewing action of a predator that may eat the prey. In the event that the shell material is one that will dissolve or corrode when placed in the sub cutaneous fat layer of a prey animal or if the shell material is porous or contains holes an optional encapsulation coating that covers the shell can be added that is insoluble in body fats to aid in the long-term stability of the system when it resides in the sub cutaneous fat layer of a prey animal.

In the event that the existing enteric coating on commercially available pills is not thick or resistant enough to give the desired dispensing delay time an enteric filler can be placed around a pill that has been loaded into a shell to provide additional enteric protection.

The enteric function of a traditional enteric coated pill can be provided by the plugs that are pressed into the ends of a tube. By changes in the size of the tube and the depth and or material of these plugs the time of drug release can be tailored to the digestive residence time of the target animal.

Furthermore, to aid in determining the position of the capsule by metal detector, x-ray, sonogram, or other imaging equipment in a dead or captured animal the shell can be made of copper, nickel, silver, gold or other non-acid sensitive metal or a more acid sensitive material can be plated with these non-acid sensitive materials when the shell is made of aluminum or other acid sensitive materials. The shell or trackers can be a radioactive material. Radio transmitters can be placed in the shell to help track prey animals or predator animals that have ingested the capsule. Further still, scientific instruments and data loggers including thermometers, pH meters, etc. can be placed in the shell to enable data recovery.

The foregoing detailed description has been given for clearness of understanding only and no unnecessary limitations should be understood therefrom. Accordingly, while one or more particular embodiments of the disclosure have been shown and described, it will be apparent to those skilled in the art that changes and modifications may be made therein without departing from the invention if its broader aspects, and, therefore, the aim in the appended claims is to cover all such changes and modifications as fall within the true spirit and scope of the present disclosure.

What is claimed is:

1. A chemical delivery capsule assembly comprising:
a drug for poisoning a target species;
an enteric coating for preventing dispensing of said drug in a stomach of an animal; and
a protective shell for preventing premature puncturing of said assembly by an animal.

2. The assembly as defined by claim 1 including an outer encapsulation.

3. The assembly as defined by claim 1 wherein said shell is perforated.

4. The assembly as defined by claim 1 wherein said shell is cupped.

5. The assembly as defined in claim 4 wherein said cupped shell includes a plug.

6. The assembly as defined in claim 4 wherein said cupped shell includes a supporting rim.

7. The assembly as defined in claim 1 wherein said shell is adjustable in size.

8. The assembly as defined in claim 1 including at least one tracer for determining a location of said assembly.

9. A targeted chemical delivery system for passing the chemical through a predator mammal and targeting a predator reptile, the system comprising:
a drug;
an enteric coating said drug, said coating having a thickness;
a mammal digestive time and a reptile digestive time, said reptile digestive time greater than said mammal digestive time; and
said thickness of said coating breaks down and releases said drug after said mammal digestive time.

10. The system as defined by claim 9 including a protective shell for preventing animal premature puncturing of said coating.

11. The system as defined by claim 9 including an outer encapsulation.

12. The system as defined by claim 10 wherein said shell is perforated.

13. The system as defined by claim 10 wherein said shell is cupped.

14. The system as defined by claim 13 wherein said shell includes a plug.

15. The system as defined by claim 13 wherein said shell includes a supporting rim.

16. A method for targeting a chemical delivery to a specific species, said method consisting of:
providing a drug capsule having an enteric coating that breaks down in intestines and releases said drug;
inserting said capsule into a bait;
releasing said bait; and
poisoning said specific species after said coating breaks down and releases said drug.

17. The method as defined by claim 16 wherein said bait is a live prey animal.

18. The method as defined by claim 17 wherein said inserting including adhering said capsule to a shaved portion of said prey animal.

19. The method as defined by claim 17 wherein said inserting including threading said capsule to a prey animal.

20. The method as defined by claim 17 further consisting of providing a tracer within said capsule and tracing said tracer.
